(19) United States
(12) Patent Application Publication
(10) Pub. No.: US 2024/0151723 A1
(43) Pub. Date: May 9, 2024

(54) METHOD FOR ESTIMATING THE TOXICITY LEVEL OF RADIATION THERAPY ON A PATIENT

(71) Applicants: Neolys Diagnostics, Entzheim (FR); Alara Group, Entzheim (FR)

(72) Inventors: Sandrine Pereira, La Bastidonne (FR); Fanny Carbillet, Ostwald (FR)

(21) Appl. No.: 18/549,001

(22) PCT Filed: Mar. 3, 2022
(86) PCT No.: PCT/FR2022/050384

§ 371 Date: Sep. 5, 2023

(30) Foreign Application Priority Data
Mar. 5, 2021 (FR) 2102157

(51) Int. Cl. G01N 33/573

(52) U.S. Cl. CPC G01N 33/573; G01N 2333/91215; G01N 2440/14; G01N 2800/52

(57) ABSTRACT

A method for estimating a toxicity level of radiation therapy on a patient comprises characterizing radiosensitivity of a cell sample from the patient to ionizing radiation to determine a first radiosensitivity factor by biochemical analysis. The first radiosensitivity factor is determined by biochemical analysis of a sample collected from the patient under examination in a basal state, to quantify a marker or a combination of markers. Dosimetric data is analyzed using data originating from treatment planning to determine a second treatment factor. The level of toxicity is determined by a combination of the first radiosensitivity factor and the second treatment factor. The method may be used to assess the risk factors of a radiation therapy treatment on a patient suffering from, for example, cancer to determine whether the patient will be able to tolerate further radiation without there being a major, or even lethal, toxicological risk.

### CROSS-REFERENCE TO RELATED APPLICATIONS

This application is a national phase entry under 35 U.S.C. § 371 of International Patent Application PCT/FR2022/050384, filed Mar. 3, 2022, designating the United States of America and published as International Patent Publication WO 2022/185015 A1 on Sep. 9, 2022, which claims the benefit under Article 8 of the Patent Cooperation Treaty of French Patent Application Serial No. 2102157, filed Mar. 5, 2021.

### TECHNICAL FIELD

The present disclosure relates to the field of oncology and more particularly to the assessment of the risk factors of a radiation therapy treatment on a patient suffering from, for example, cancer in the head or neck region, in order to determine whether the patient will be able to tolerate further radiation without there being a major, or even lethal, toxicological risk.

### BACKGROUND

Radiation therapy (RT) is one of the most frequently used and most effective treatments against cancer. Like any anti-cancer treatment, the therapeutic benefit of radiation therapy is weighed against the potential adverse effects (or toxicity) in normal tissue. Therefore, it is of crucial importance to grade and precisely report on the undesirable effects of radiation therapy, in order to improve the quality of the RT and to search for effective treatments.

The total dose of ionizing radiation delivered to the tumor is a major factor of locoregional control. The increase in this dose to tumor volumes is most often correlated with an increase in the dose to healthy tissues.

About 1 to 15% of patients treated by radiation therapy for a cancer have a tissue reaction (such as dermatitis or proctitis) that may compromise the correct progress of the treatment insofar as it can lead the physician to decide to halt the radiation therapy treatment before the end of the intended protocol. Furthermore, this tissue reaction is an indicator of particularly high sensitivity of the patient to ionizing radiation. Thus, the radiation therapy treatment, even if it is interrupted upon the appearance of the first visible tissue signs, can increase the post-treatment morbidity or mortality of patients, not only because the cancer that it was intended to treat was not able to be completely eradicated due to the premature halting of the treatment, but also due to the collateral damage of the healthy tissues induced by the radiation itself.

The planning of external radiation therapy must therefore take into account constraints of dose to the organs at risk. The delivery of a highest dose generates more toxicity, both early and late.

This toxicity must be recorded in a prospective manner by the practitioners.

The recording and grading must be done in a simple, reproducible and sensitive manner in order to obtain a precise and suitable assessment for each organ, and the “CTCAE” reference system (Common Terminology Criteria for Adverse Events) developed by the National Cancer Institute constitutes a recognized indicator to enlighten the radiotherapist on the suitability of initiating further radiation. It uses a scale of grades ranging from 1 to 5 corresponding, in increasing order, to the qualifiers Mild, Moderate, Severe, Life-threatening, Death.

To objectify the appropriate criterion for a patient, it is necessary to have reliable analysis tools, in order to avoid abandoning further radiation for a patient who could tolerate it, and to forgo further radiation for a patient in whom it would lead to fatal consequences.

It is therefore desirable to have a predictive test method in order to be able to determine the maximum cumulative dose that a given patient can receive without risk. This question is first asked in radiation therapy in a context of high ionizing doses.

Several scientific articles concerning toxicity resulting from radiation therapy are known in the state of the art.

The article by De Ruyck K. et al.: “A predictive model for dysphagia following IMRT for head and neck cancer: Introduction of the EMLasso technique,” Radiother. Oncol., vol. 107, no. 3, Apr. 22, 2013, pages 295-299 relates to the risk of dysphagia, especially after radiation therapy combined with chemotherapy. Dysphagia is a clinical sign that can result from radiation with excessive doses but is not directly representative of the general toxicity level. Indeed, the toxicity level can be reflected by other clinical syndromes, and taking into account the risk of dysphagia does not constitute a relevant indicator.

This document also proposes a method based on genotyping of a blood sample by polymorphism analysis and not by determining the amount of phosphorylated ATM protein pATM in the basal state in the sample. The protocol is based on a genomic analysis, which does not appear to directly predict individual radiosensitivity.

The article by Kang J. et al.: “Genomics models in radiotherapy: From mechanistic to machine learning,” Md. Phys., vol. 47, no. 5, May 15, 2020, pages e203-e217 also describes a genomics-based approach. The article proposes an examination of radiogenomics modeling frames and efforts in the field of genomics-guided radiation therapy in order to create clinical tests of radiosensitivity in normal tissues or tumors. FIG. 2a of the article relates to a theoretical example of the combination of NTCP models with a biological variable. The biological variable may not be a radiosensitivity factor within the meaning of the claimed disclosure.

The article by El Naqa I. et al.: “Radiogenomics and radiotherapy response modeling,” Phys. Med. Biol., vol. 62, no. 16, Aug. 1, 2017, pages R179-R206 also describes a genomics-based approach. It does not relate to the prediction of a radiosensitivity factor but rather to a genomics study that does not appear to directly predict individual radiosensitivity.

The article by Orlandi E. et al.: “Multivariable model for predicting acute oral mucositis during combined IMRT and chemotherapy for locally advanced nasopharyngeal cancer patients.” Oral Oncol., vol. 86, Oct. 11, 2018. pages 266-272 describes NTCP models in ORL cancers but only with dosimetric data and no biological variables.

The article by Werbrouck J. et al.: “Acute normal tissue reactions in head-and-neck cancer patients treated with IMRT: Influence of dose and association with genetic polymorphisms in DNA DSB repair genes,” Int. J. Radiation Oncol. Biol. Phys., vol. 73, no. 4, Feb. 26, 2009, pages 1187-1195 does not relate to the prediction of a radiosensitivity factor but rather to a genomics study that does not appear to directly predict individual radiosensitivity.

The article by Granzotto A. et al.: “Influence of nucleoshuttling of the ATM protein in the healthy tissues response to radiation therapy: Toward a molecular classification of human radiosensitivity,” Int. J. Radiation Oncol. Biol. Phys., vol. 94, no. 3.14 Nov. 2015, pages 450-460 suggests analyzing human radiosensitivity based on 3 groups: radioresistance (group I); moderate radiosensitivity caused by a delay in nucleoshuttling of the ATM, which includes patients in an operating room (group II); and hyperradiosensitivity caused by a rough DSB repair defect, which comprises fatal cases (group III). It provides a protocol based on the kinetics of marker protein degradation during additional radiation applied to a sampled tissue. It provides for carrying out immunofluorescence experiments on a collection of skin fibroblasts from 12 radioresistant patients, 5 hyperradiosensitive patients and 100 OR patients irradiated with 2 Gy. The numbers of micronuclei, of yH2AX foci and pATM that reflect the various steps of the recognition and repair of DNA double-stranded breaks (DSB) were assessed from 10 minutes to 24 hours after radiation and traced as a function of the degrees of severity established by the Common Terminology Criteria for Adverse Events.

The solutions of the prior art based on the quantification of biomarkers were not totally satisfactory and notable variations are observed from one patient to another, resulting in overly favorable predictions in certain cases, which can lead to negative outcomes for certain patients, and overly pessimistic predictions in other cases, which deprive the patient of continued radiation therapy treatment that would have made it possible to reduce the cancer cells.

The object of the present disclosure is to provide a better quality risk factor estimation, customized for each patient, with a lower margin of uncertainty.

### BRIEF SUMMARY

In order to address these disadvantages, the present disclosure relates to a method for estimating the level of toxicity of a radiation therapy treatment on a patient. The method includes a step of characterizing radiosensitivity of a cell sample to ionizing radiation, the cell sample having been obtained from cells collected from a patient, in order to determine a first radiosensitivity factor by a biochemical analysis. The first radiosensitivity factor is determined by a method for biochemical analysis of a sample collected from the patient under examination in a basal state, in order to quantify a marker or a combination of markers. The method further includes a step of analyzing dosimetric data using data originating from treatment planning, in order to determine a second treatment factor, and the level of toxicity is determined by a combination of the first radiosensitivity factor and a second treatment factor.

The term “biochemical analysis” is understood to mean, within the meaning of the present patent, all the molecular biology analyses, carried out using a biological sample taken from the patient making it possible to determine a radiosensitivity factor.

The present disclosure does not relate to methods based on genetic analyses alone. According to a first variant, the radiosensitivity factor is determined on fibroblasts originating from a sample of connective tissue of the patient.

According to a second variant, the radiosensitivity factor is determined on fibroblasts originating from a sample of the skin of the patient.

According to a third variant, the radiosensitivity factor is determined on lymphoblasts/lymphocytes originating from a blood sample of the patient.

Advantageously, the radiosensitivity factor is determined by determining from the total fractions of the cell sample of the collection the amount of phosphorylated ATM protein (pATM) in the basal state.

According to one example embodiment, the toxicity level is determined by an adjustment of the bivariate logistic regression models, comprising:
(a) the result of the pATM test on the blood lymphocytes; and
(b) the prediction using dosimetric models based on the treatment planning dosimetric data for toxicities of grade ≥3 or grade ≥2.

### BRIEF DESCRIPTION OF THE DRAWINGS

The present disclosure will be better understood on reading the following description, illustrated by the accompanying drawings relating to non-limiting embodiments, wherein:

FIG. 1 depicts the results on the development of inflammation of the mucosa in radiosensitive patients having a CTCAE grade greater than or equal to 2.

FIG. 2 depicts the results on the development of inflammation of the mucosa in radiosensitive patients having a CTCAE grade greater than or equal to 3.

FIG. 3 depicts the results on dysphagia in radiosensitive patients having a CTCAE grade greater than or equal to 2.

FIG. 4 depicts the results on dysphagia in radiosensitive patients having a CTCAE grade greater than or equal to 3.

FIG. 5 depicts the results on acute toxicity and late toxicity in patients.

FIGS. 6A-6D depict the results on dysphagia and mucositis for patients with toxicities having a CTCAE grade greater than or equal to 2 and 3.

FIG. 7 depicts the results for patients suffering from prostate cancer and having late toxicities with a CTCAE grade greater than or equal to 2.

### DETAILED DESCRIPTION

General Principle

The object of the present disclosure is to offer physicians an objective and reliable indicator that is representative of the ability of a patient to tolerate radiation in the context of radiation therapy. This indicator is determined based on the results of at least two protocols:
an in vitro radiosensitivity analysis carried out on a biological sample from the patient, in order to establish a radiosensitivity factor on the sample collected with or without application of radiation after collection; and
a dosimetric analysis of the treatment planning system (TPS) established to prepare a radiation treatment plan for radiation therapy so as to establish a treatment factor.

These two factors are combined to establish a predictive indicator of the toxicological risk level in the “CTCAE” (Common Terminology Criteria for Adverse Events) reference frame.

They can be completed by other analyses, including by genetic analyses combined with the above-mentioned analyses.

Determining the Radiosensitivity Factor

The first factor, referred to as radiosensitivity factor, is determined by a known method for biochemical analysis of a sample from the patient under examination in order to quantify a marker or a combination of markers, especially of proteins of the kinase family, such as ATM and ATR.

It is known that the question of tissue sensitivity to ionizing radiation is linked to the mechanisms for DNA damage repair. Indeed, at the cellular level, ionizing radiation can break certain types of chemical bonds, generating free radicals (in particular, by peroxidation) and other reactive species that cause DNA damage. DNA damage due to endogenous or exogenous attacks (such as ionizing radiation and free radicals) can result in different types of DNA damage based especially on the deposited energy: base damage, single-strand breaks and double-strand breaks (DSB). Unrepaired DSBs are associated with cell death, toxicity and more specifically, radiosensitivity. Incorrectly repaired DSBs are associated with genomic instability, mutagenic phenomena and a predisposition to cancer. The body has repair systems specific to each type of DNA damage. As regards DSBs, mammals have two main repair modes: repair by suture (strand ligation) and repair by recombination (insertion of a homologous or non-homologous strand).

It is also known that tissue sensitivity to ionizing radiation varies greatly from one organ to another and from one individual to another. Even outside of certain rare cases of extreme radiosensitivity, the genetic origin of which appears to be proven, it is believed that radiosensitivity is generally the result of a genetic predisposition: it is therefore specific to an individual.

By way of non-limiting example, the radiosensitivity factor is determined using a healthy tissue sample, preferably fibroblasts or lymphoblasts/lymphocytes. The first ones are preferably collected from the connective tissue or the skin of an individual and the latter from the blood. This sample can be collected by biopsy or by taking a blood sample.

Example of a Protocol for Determining the Radiosensitivity Factor

The radiosensitivity factor can be determined using a kit to characterize the radiosensitivity to ionizing radiation of a cell sample of an individual. This kit comprises:
a) means for extracting the total fractions of the cell sample, that is to say, reagents such as a lysis buffer, which are known to a skilled person,
b) means for determining in the fractions of the cell sample the amount of phosphorylated ATM protein pATM in the basal state (in the state in which the protein is found in the cell, without external modification such as radiation), such as a plate covered with a specific anti-pATM antibody for carrying out an ELISA test.

The basal state of a protein corresponds to the expression level of the protein in the absence of activation or repression induced by cellular oxidative stress but also in the absence of damage to the cell by radiation.

The total fraction of a cell sample obtained after total lysis contains all the proteins of the cell, including the nuclear and cytoplasmic proteins and more particularly the pATM proteins.

The expression of the ATM protein can also be determined upstream by quantifying the expression of the messenger RNA coding for the protein by genomic analysis techniques.

Concept of “Total Traction” and “Basal,” and Other Types of Markers

The operator collects a cell sample from the patient, such as a blood sample comprising lymphoblasts or lymphocytes.

The blood components can be separated by centrifugation on a cushion consisting of a copolymer of saccharose (sucrose) and epichlorohydrin, FICOLL®. After centrifuging under suitable conditions known to the skilled person, the blood components are separated by density. The lymphocytes thus purified are re-dispersed in a suitable culture medium, preferably in a culture medium of the RPMI 1640 type supplied by the company Gibco and comprising 10% of fetal calf serum and 1% of penicillin/streptomycin. These lymphocytes in the basal state thus obtained are then used as such, without being irradiated (absorbed dose 0 Gy). The biological material is subjected to total lysis by any appropriate means. The isolation and capture of the phosphorylated ATM proteins (which are thus active) in each of the total, cytoplasmic and/or nuclear fractions is carried out by ELISA test, via the use of plates covered with a specific anti-pATM antibody. In each fraction, the amount of pATM is then analyzed by luminescence at 450 nm by way of a plate reader. All of the techniques indicated are known to a skilled person.

Determining the Genetic Factor

The genetic factor is determined by a known method for sequencing the DNA extracted from a sample of the patient under examination in order to qualify markers or a combination of markers, especially of DNA polymorphisms.

It is known that certain DNA polymorphisms can be present in individuals who develop toxicity following radiation. It is also known that tissue sensitivity to ionizing radiation is highly variable from one organ to another and from one individual to another.

By way of non-limiting example, the genetic factor is determined using a healthy tissue sample, preferably lymphoblasts/lymphocytes or fibroblasts. The first ones are preferably collected from the blood and the latter from the connective tissue or the skin of an individual. This sample can be collected by blood sampling or biopsy.

Example of a Protocol for Determining the Genetic Factor

The genetic factor can be determined by a procedure that is well-known to a skilled person, consisting in carrying out an analysis by genotyping the DNA and including:
a) means for extracting the DNA from the cell sample, that is to say from reagents such as a lysis buffer known to the skilled person,
b) means for determining in the DNA from the cell sample the genotype of certain polymorphisms in the basal state (state in which the DNA is found in the cell, without external modification such as radiation), by using sequencing reagents known to a skilled person and a sequencer.

The basal state corresponds to the absence of damage to the DNA of the cell by radiation. The genotyping information is then analyzed in silico to determine the genotypes of the polymorphisms tested for each individual. A multivariate logistic regression is then used to determine a polygenic risk score. The calculation of this score is detailed in the following article:
Choi S W, Mak T S, O'Reilly P F, Tutorial: a guide to performing polygenic risk score analyses. Nat Protoc. 2020 September; 15(9):2759-2772. doi: 10.1038/s41596-020-0353-1. Epub 2020 Jul. 24. PMID: 32709988; PMCID: PMC7612115.

Determining the Treatment Factor

The treatment factor is determined using the data available from the treatment planning system (TPS), extracted from the software for preparing the radiation treatment plan for the radiation therapy of a patient under examination. TPS has become an essential tool for modern radiation therapy. It makes it possible, using the medical prescription, the images and the anatomical volumes recovered by the medical physics team to prepare the treatment plan, to put in place the most suitable treatment ballistics (energy of the beams; number, shape and incidence of the fields; intensity modulation or not, etc.). Often, several tests are carried out. The dose is calculated using an algorithm that models the deposition of energy in the material, according to the laws of interaction of the particles involved. It enables:
3D viewing of the anatomical images of the patient (multimode: MRI, scanner and PET).
Optional registration of two or more independent sets of images.
Delineation of the target volumes and volumes at risk.
Creation of treatment ballistics (positioning of the beams, choice of their energy, shape, etc.).
3D calculation of the dose in the patient, by virtue of algorithms that model the deposition of energy of the particles involved in the tissues.
Quantitative visualization of the dose deposition in the organs in the form of a dose-volume histogram (DVH). The precise operation of this software requires significant parameterization.
The calculation of the dose in the patient, using scanner images, requires knowing the conversion curve of Hounsfield Units (HU, information contained in the scanner images) to relative electron density (information directly linked to the composition of the tissues). This curve is measured for a given scanner and voltage, in a “phantom” containing inserts of different electron densities, in order to cover all the electron densities available in the human body. The calculations performed by the TPS take into account the properties of the accelerator: dose rate, energy, homogeneity and symmetry of the beams, filtration, collimation accessories, etc., which are the elements for which a precise description must be given to the TPS. This parameterization is directly linked to the step of acceptance of a treatment machine described elsewhere.

Once parameterized for the available accelerators, the TPS makes it possible to plan the treatments. Using volume images of the patient and the volumes to be treated as well as OARs delineated by the radiation oncologist, the dosimetrist programs the treatment beams with the correct parameters: energy, dimensions, orientation, dynamic filter, etc., in order to obtain the best possible dose distribution for the treatment. The dose calculation is based on algorithms capable of simulating the interaction of photons and electrons in the tissues, for example, by the Monte Carlo simulation code.

The treatment factor calculated using data from the TPS software consists, for example, of the following:
the cumulative dose (in Gy) over the entire radiation therapy treatment received by a healthy organ in the vicinity of the tumor zone
the maximum dose (in Gy) received by a healthy organ in the vicinity of the tumor zone during one radiation therapy session
the distribution of doses on the healthy organs in the vicinity of the tumor zone
and more generally, any dosimetric indicator representative of the radiation treatment of the patient under examination.

Combination of the Treatment and Radiosensitivity Factors

Combined mathematical models of radiosensitivity factor and treatment factor were developed by adjusting bivariate logistic regression models, comprising:
(a) the result of the pATM test on the blood lymphocytes (using the previously determined dichotomic classification of patients as radiosensitive vs radioresistant, with one binary variable, YES=radiosensitive, NO=radioresistant) and
(b) the prediction using dosimetric models based on treatment planning dosimetric data for toxicities of grade ≥3 or grade ≥2.

Such dosimetric models are proposed in the following articles:
Orlandi, E., Iacovelli, N. A., Rancati, T., Cicchetti, A., Bossi, P., Pignoli, E., et al. “Multivariable model for predicting acute oral mucositis during combined IMRT and chemotherapy for locally advanced nasopharyngeal cancer patients.” Oral Oncol 2018, 86, 266-272.
Cavallo, A., Rancati, T., Cicchetti, A., Iacovelli, N. A., Palorini, F., Fallal, C. et al. “Development of multivariable models for acute toxicities in nasopharyngeal cancer radiotherapy Radiother,” Oncol. 2017, 123, S858-S859.

The predictive value of the combination of treatment and radiosensitivity factors is assessed using an ROC curve analysis, the determination of the AUC (Area Under Curve) and the assessment of probabilities (Chi-squared Test).

Results Observed

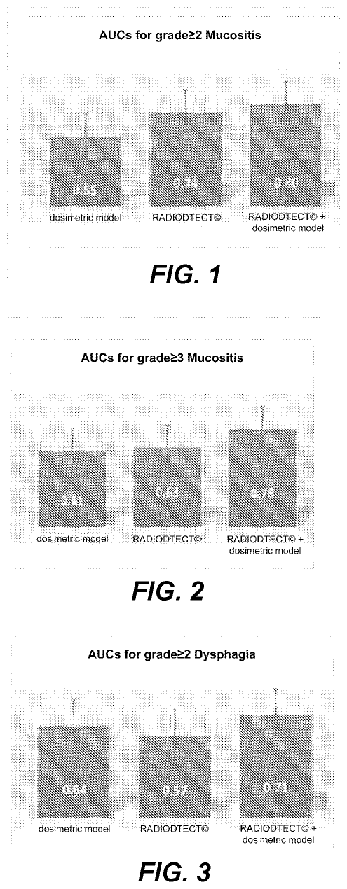

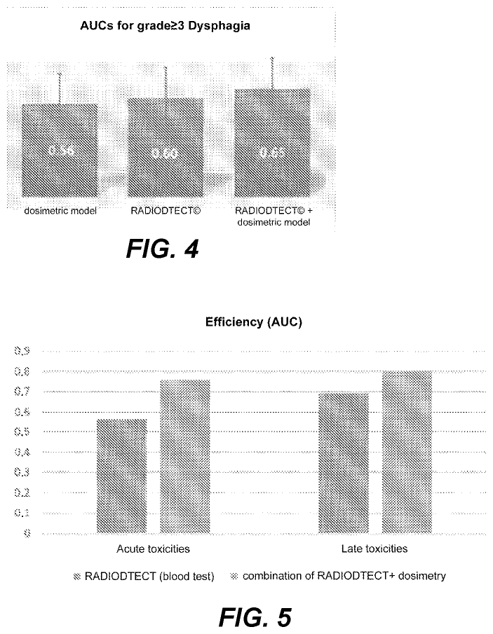

FIGS. 1 to 6D show the correlation between the predictive indicators and the diseases that actually appeared in a cohort of patients who underwent radiation therapy against head and neck cancer (HNC) observed in several clinical trials.

The predictive indicators were determined for the tests illustrated by FIGS. 1 to 4:
a) Using a dosimetric model (left-hand column)
b) Using a radiosensitivity factor obtained based on quantifying the phosphorylated ATM protein in the lymphocytes (middle column)
c) Using a combination of a treatment factor and a radiosensitivity factor combined in accordance with the present disclosure (right-hand column).

The predictive indicators were determined for the tests illustrated by FIG. 5:
a) Using a radiosensitivity factor based on quantifying the phosphorylated ATM protein in the lymphocytes (left-hand column)
b) Using a combination of a treatment factor and a radiosensitivity factor combined in accordance with the present disclosure (right-hand column).

53 patients were examined in order to assess the predictive capacity of RDT for acute toxicity of grade 3 (G3). The patients were treated in a “radical” framework with post-operative radiation therapy and were assessed in a prospective manner for the toxicity determined by the CTCAE grade at the start and once a week during the radiation therapy. The reality of the pathological consequences was determined at least 6 months after the end of the radiation therapy.

67 patients were included in the validation cohort. They were monitored in a prospective manner for the toxicity score in the same way as the training cohort. These points were also assessed for late toxicity, with monitoring for up to three years. The tests in the validation population were carried out blindly.

The analysis of the validation population comprised assessing the predictive value of the indicator determined according to the method that is the subject of the present disclosure.

The tests show that, in all cases, the predictive quality of the indicator determined according to the present disclosure is greater than the indicators known in the state of the art.

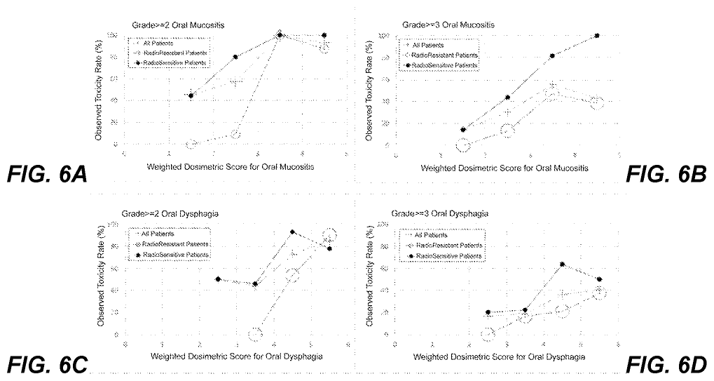

The predictive indicators determined for the tests are illustrated by FIGS. 6A-6D using a combination of a treatment factor (based on dosimetric data) and a combined radiosensitivity factor in accordance with the present disclosure. The patients were classified into two classes—radiosensitive and radioresistant—according to the combination between the radiosensitivity factor and the treatment factor.

In this study, fourteen patients out of 101 (13.9%) have acute oral mucositis of grade 1, 38 of grade 2 (37.6%), 39 of grade 3 (38.6%) and 3 of grade 4 (3%). Twenty-one patients (20.8%) have acute dysphagia of grade 1, 39 of grade 2 (38.6%), 33 of grade 3 (32.7%) and 2 of grade 4 (2%).

The patients were treated either with post-operative radiation therapy, or by a combination of chemotherapy and radiation therapy and were assessed in a prospective manner for the toxicity determined by the CTCAE grade at the start and once a week during the radiation therapy. The reality of the pathological consequences was determined at least 6 months after the end of the radiation therapy.

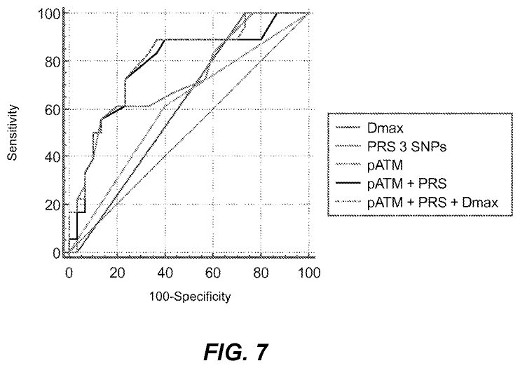

FIG. 7 shows the correlation between the predictive indicators and the late toxicities that appeared in a cohort of patients who underwent radiation therapy against prostate cancer observed in a clinical trial.

The predictive indicators were determined (ROC curves):
d) Using a dosimetric model (referred to as Dmax)
e) Using a radiosensitivity factor obtained based on quantifying the phosphorylated ATM (pATM) protein in the lymphocytes
f) Using the calculation of the polygenic risk score (PRS) based on genotyping information of 3 polymorphisms already described in the literature (rs11122573 from Kerns et al, J Natl Cancer Inst 2020, rs2293054 and rs845552 from De Langhe et al, RO 2014)
g) Using a combination of a treatment factor with a radiosensitivity factor and a polygenic risk factor combined in accordance with the present disclosure.

1. A method for estimating a toxicity level of radiation therapy on a patient, comprising a step of characterizing radiosensitivity of a cell sample to ionizing radiation, the cell sample having been obtained from cells collected from a patient, to determine a first radiosensitivity factor by a biochemical analysis, wherein the first radiosensitivity factor is determined by a method for biochemical analysis of a sample collected from the patient under examination in a basal state, to quantify a marker or a combination of markers, and a step of analyzing dosimetric data using data originating from treatment planning, to determine a second treatment factor, and wherein the level of toxicity is determined by a combination of the first radiosensitivity factor and the second treatment factor.

2. The method of claim 1, wherein the determination of the radiosensitivity factor representative of the ability of a patient to tolerate further radiation in the context of radiation therapy is determined based on the results of at least two protocols:
an in vitro radiosensitivity analysis carried out on a biological sample from the patient, to establish a radiosensitivity factor on the sample collected with or without application of radiation after collection; and
a dosimetric analysis of the treatment planning system (TPS) established to prepare a radiation treatment plan for radiation therapy so as to establish a treatment factor.

3. The method of claim 1, wherein the determination of the factor is further based on a protocol for determining a genetic factor.

4. The method of claim 1, wherein the radiosensitivity factor is determined by determining an amount of phosphorylated ATM protein pATM of the sample in the basal state.

5. The method of claim 1, wherein the radiosensitivity factor is determined on fibroblasts originating from a sample of connective tissue of the patient.

6. The method of claim 1, wherein the radiosensitivity factor is determined on fibroblasts originating from a sample of skin of the patient.

7. The method of claim 1, wherein the radiosensitivity factor is determined on lymphoblasts/lymphocytes originating from a blood sample of the patient.

8. The method of claim 1, wherein the toxicity level is determined by adjusting bivariate logistic regression models, which comprises:
(a) the result of the pATM test on the blood lymphocytes; and
(b) the prediction using dosimetric models based on treatment planning dosimetric data for toxicities of grade ≥3 or grade ≥2.
